(19) United States
(12) Patent Application Publication
(10) Pub. No.: US 2007/0089741 A1
(43) Pub. Date: Apr. 26, 2007

(54) Method and apparatus for changing the concentration of a target gas at the blood compartment of a patient's lung during artificial ventilation

(76) Inventors: Stephan Bohm, Lauenburg an der Elbe (DE); Gerardo Tusman, Buenos Aires (AR); Christoph Manegold, Idstein (DE)

Correspondence Address: SHLESINGER, ARKWRIGHT & GARVEY LLP, Suite 600, 1420 King Street, Alexandria, VA, 22314, US

(21) Appl. No.: 11/583,952

(22) Filed: Oct. 20, 2006

Related U.S. Application Data
(63) Continuation of application No. PCT/EP05/04353, filed on Apr. 22, 2005.

(30) Foreign Application Priority Data
Apr. 23, 2004 (EP) 04 009 688.5
Oct. 22, 2004 (EP) 04 025 174.6
Feb. 10, 2005 (EP) 05 002 840.6

(51) Int. Cl. A61M 15/00; A62B 7/00

(52) U.S. Cl. 128/203.12; 128/200.24

(57) ABSTRACT

The invention refers to a method and apparatus for changing the concentration of a target gas at the blood compartment of a patient's lung from an actual target gas concentration to a desired target gas concentration during artificial ventilation with an inspiratory gas composition by a respirator being controlled via a set of ventilation parameters. In order to decrease the negative effects of general anaesthesia during artificial ventilation even further, the method according to the invention comprises the following steps: a) ventilating the lung in a first ventilation stage, and b) ventilating the lung in a second ventilation stage in which alveolar recruitment is promoted.

### FIELD OF THE INVENTION

The invention refers to a method and an apparatus for changing the concentration of a target gas at the blood compartment of a patient's lung from an actual target gas concentration to a desired target gas concentration during artificial ventilation with an inspiratory gas composition by a respirator being controlled via a set of ventilation parameters.

### BACKGROUND OF THE INVENTION

The main function of the lung is gas exchange between atmospheric and blood gases where oxygen is absorbed into the blood and carbon dioxide, a product of body metabolism, is eliminated.

For maintaining this functioning, a lung needs to keep its normal morphology. Any 3D-morphological change will be related to an abnormal gas ventilation and blood perfusion distribution inside it. As a consequence, the alveolar-capillary membrane i.e. the lung zone where gas exchange takes place, cannot work optimally. In other words, any distortion of the normal ventilation and perfusion relationship affects normal gas exchange and a single patient will suffer from hypoxemia (decrease in arterial oxygenation).

Therefore, a perfect ventilation and perfusion relationship (V/Q) inside the alveoli is needed for a normal lung function. Any variation from the ideal value of 1 causes a deterioration of the gas exchange due a mismatching between these two functions.

During anaesthesia the patient's lung is filled with an inspiratory gas composition consisting of a fresh gas and possibly a fraction of re-breathed gas. The fraction of re-breathed gas is added only in semi-closed or closed circle breathing systems, whereas in open breathing systems the inspiratory gas composition consists purely of fresh gas. The fresh gas is composed of the target gas, i.e. the anaesthetic agent, oxygen and a carrier gas, i.e. nitrous oxide, helium or air. Inhalatory anaesthetic agents like halothane, isofluorane and sevofluorane are widely used in anaesthesia. These vapors enter the human beings by means of ventilation, delivered by an anaesthesia machine. The inhalatory agents reach the blood by diffusion through the alveolar-capillary membrane and are transported by the blood to the central nervous system. Diffusion is a passive transport through a membrane due to a partial pressure gradient. This means that inhalatory anaesthetic molecules go from the side with higher partial pressure to the side of the membrane with lower pressure. Firstly, during anaesthesia induction, where tissue anaesthetic concentration is zero, anaesthetic molecules go from the alveolar compartment (high concentration) to the blood (low concentration). In the opposite way, at the end of surgery when anaesthetic agent is withdrawn, anaesthetic concentration is higher at the blood compartment so that molecules follow an inverse way and are eliminated by breathing.

However, general anaesthebia and mechanical ventilation have a negative effect on the respiratory system. Thus both, respiratory mechanic and gas exchange through the alveolar-capillary membrane, deteriorate within 5 minutes from anaesthesia induction. This pathologic phenomenon is caused by a loss of gas volume inside the lungs due to closing of normally aerated lung regions, known as “lung collapse”.

Recently, ventilatory recruitment maneuvers have been developed to solve the “lung collapse” problem in healthy and sick lungs. Recruitment maneuvers consist of a controlled increment in airway pressure until a point where the airway opening pressure is reached (the opening airway pressure of the lung is the airway pressure at which the closed units of the lung start opening). Afterwards, mechanical ventilation reassumes baseline ventilation with a level of positive end-expiratory pressure (PEEP) higher than the lung's closing pressure (i.e. airway pressure where opened units start closing, again).

In Tusman et.al.: “Alveolar Recruitment Strategy improves arterial oxygenation during general anaesthesia”, British Journal of Anaesthesia 82(1) 8-13(1999), and in Tusman et. al.: “Alveolar recruitment strategy increases arterial oxygenation during one-lung ventilation”, Annals of Thoracic Surgery 73:1204-1209 (2002) and in Tusman et.al.: “Effects of recruitment maneuver on atelectasis in anesthetized children”, Anesthesiology 98:14-22 (2003) and in Tusman et.al.: “Lung recruitment improves the efficiency of ventilation and gas exchange during one-lung ventilation anesthesia”, Anesthesia Analgesia 98: 1604-1609 (2004) and in Tusman et.al.: “Deadspace analysis before and after lung recruitment”, Canadian Journal of Anesthesia 51:718-722 (2004) a recruitment maneuver is described which is used systematically for anesthetized patients. This maneuver has been useful to normalize lung volumes and gas exchange. Taking into account the above explanations, the alveolar recruitment strategy normalizes gas exchange because it improves ventilation and perfusion distribution within lungs, restoring an adequate V/Q relationship.

By way of an example, FIG. 1 shows a typical recruitment maneuver in detail. As shown in FIG. 1, the recruitment maneuver is carried out on the basis of a pressure controlled ventilation and uses two pressure levels, namely the peak inspiratory pressure (PIP) during inspiration and the positive end-expiratory pressure (PEEP) during expiration. Before the final recruitment maneuver takes place, the alveolar opening pressure and the alveolar closing pressure have to be identified. In a first step (step 1), PIP and PEEP are stepwise increased by means of an incremental limb until the alveolar opening pressures have been detected with regard to PIP and PEEP (steps 2 and 3). The alveolar opening pressure with regard to PIP is usually about 40 cmH₂O in normal lungs and in the range of 55-60 cmH₂O in sick lungs. After a successful alveolax opening, a decremental limb or stepwise decrease of PIP and PEEP is done (step 4) to determine the alveolar closing pressure (step 5). After having identified the pressures for alveolar opening and alveolar closing, the final recruitment maneuver (step 6) is done with these new target pressures over 10 breaths and PEEP is set above the alveolar closing pressure to avoid pulmonary re-collapse. For example, PEEP is set 2 cmH₂O above the alveolar closing pressure, i.e.
PEEP=PEEP_{close}+2 cmH₂O

This alveolar recruitment strategy is used to ventilate patients with normal lungs as well as those with an acute lung disease in order to keep the lung open in case of a lung collapse. In other words, the alveolar recruitment strategy is applied for improving the gas exchange characteristic of a lung and thus to improve the mechanical behaviour of the patient's lung during artificial ventilation.

However, despite these efforts there remain various negative effects on the patient's body and in particular on the patient's respiratory system due to general anaesthesia.

### OBJECTS AND SUMMARY OF THE INVENTION

Therefore, it is an object of the invention to decrease the negative effects of general anaesthesia during artificial ventilation even further.

This object is solved by a method for changing the concentration of a target gas at the blood compartment of a patient's lung from an actual target gas concentration to a desired target gas concentration during artificial ventilation with an inspiratory gas composition by a respirator being controlled via a set of ventilation parameters, by varying the fraction of the target gas supplied to the inspiratory gas composition, and/or the fraction of the re-breathed gas supplied to the inspiratory gas composition, and/or the set of ventilation parameters being responsible for the ventilated lung volume, wherein
a) the lung is ventilated in a first ventilation stage by setting a fraction of target gas, a fraction of re-breathed gas and a set of ventilation parameters, wherein said setting results in the actual target gas concentration, and
b) the lung is ventilated in a second ventilation stage in which at least once
   the set of ventilation parameters is varied for yielding an increased ventilated lung volume compared to the first ventilation stage and
   on the basis of the increased ventilated lung volume the fraction of target gas and/or the fraction of re-breathed gas is varied such that the target gas concentration is changed towards the desired target gas concentration.

Furthermore, the above mentioned object is solved by an apparatus for changing the concentration of a target gas at the blood compartment of a patient's lung from an actual target gas concentration to a desired target gas concentration during artificial ventilation with an inspirator gas composition by a respirator being controlled via a set of ventilation parameters, comprising target gas varying means for varying the fraction of the target gas supplied to the inspiratory gas composition, re-breathed gas varying means for varying the fraction of the re-breathed gas supplied to the inspiratory gas composition, parameter varying means for varying the ventilation parameters being responsible for the ventilated lung volume, and controlling means for controlling the target gas varying means, the re-breathed gas varying means and the parameter varying means such that
a) the lung is ventilated in a first ventilation stage by setting a fraction of target gas, a fraction of re-breathed gas and a set of ventilation parameters, wherein said setting results in the actual target gas concentration, and
b) the lung is ventilated in a second ventilation stage in which at least once
   the set of ventilation parameters is varied for yielding an increased ventilated lung volume compared to the first ventilation stage and
   on the basis of the increased ventilated lung volume the fraction of target gas and/or the fraction of re-breathed gas is varied such that the target gas concentration is changed towards the desired target gas concentration.

The invention makes use of the fact that gas exchange during ventilation can be improved for all inhaled gases including anaesthetic agents when the ventilated lung volume is temporarily increased. The invention has recognized that the exchange of anaesthetic agents at the alveolar-capillary membrane can be improved on the basis of an increased ventilated lung volume, e.g. during and after an alveolar recruitment strategy due to normalization in V/Q relationship. This fact has an important clinical and economical meaning. For the clinical world, an improvement in gas exchange efficiency allows a faster anaesthesia induction, adjustment and emergence. For the economical world, an improved efficiency of gas exchange means that a lower amount of anaesthetic agents is needed for a single anaesthesia, thus decreasing hospital costs.

According to the invention, it has to be distinguished between a first ventilation stage and a second ventilation stage for changing the concentration of a target gas at the blood compartment from an actual target gas concentration to a desired gas concentration. The steady state of the first ventilation stage corresponds to the actual target gas concentration of the blood compartment. The aim is now to change the concentration of the target gas at the blood compartment towards the desired target gas concentration during the second ventilation stage. According to a preferred embodiment of the invention, during the second ventilation stage the alveolar recruitment strategy is applied wherein at the same time the inspiratory gas composition is controlled such that the second ventilation stage yields a change of the actual target gas concentration of the blood compartment towards the desired target gas concentration of the blood compartment.

However, one technical difficulty of alveolar recruitment strategy regarding inhalatory anaesthetic delivery to the patients is a dilution effect. The alveolar recruitment strategy demands a high-flow to fill the gain of lung volume (recruited volume) while the target airway pressures are reached. Application of this additional volume is hindered due to the restricted capacity of the tidal volume generating modules (bag-in-bellow, bag-in-bottle, piston driven ventilator) of traditional anaesthesia machines. Additionally, dilution effects can be caused by the re-breathed gas in a semi-closed or closed circle system or by extensive use of the oxygen flush function. Thus, an amount of a volume of gas without anaesthetic agents enters into the lung and into the anaesthetic circuit, diluting the anaesthetic gas concentration at the alveolar-capillary membrane. Obviously, this dilution effect wastes anaesthetic agents and increases the chance of an inadvertent recovery or awareness of the patient.

Therefore, the invention controls the inspiratory gas composition during the second ventilation stage such that the change from the actual target gas concentration towards the desired target gas concentration is supported. In particular, when a sudden increase of the ventilated lung volume occurs due to the increase of the peak inspiratory pressure and the positive end-expiratory pressure, the increased volume is filled with a gas which yields a change of the actual target gas concentration of the blood compartment towards the desired target gas concentration of the blood compartment. More specifically, it has to be ensured that the additional gas which is filled in the increased lung volume is of the type of the desired target gas concentration which has to be achieved in the blood compartment.

In practice, the invention can be realized by switching between the usual closed ventilation system and an adapted open ventilation system. In the first ventilation stage, a closed ventilation system can be applied. This means, that re-breathed gases are re-circulated in the system which makes the system cost-efficient because anaesthetic agents can be re-used. However, it also has to be observed that a re-breathing might cause a dilution effect so that the fraction of the specific target gas supplied to the inspiratory gas composition might vary within a certain range.

On the other hand, in the second ventilation stage an open ventilation system is more appropriate for a well controlled variation of the fraction of target gas. It has to be observed that due to the increased lung volume a closed ventilation system in the second ventilation stage causes a considerable dilution effect when supplying the additional gas (usually air) to the increased lung volume. However, having an open ventilation system it is possible to fill the increased lung volume with the appropriate gas, e.g. the desired target gas itself. At the same time, the expired gases coming from the patient can be discarded in order not to dilute the inspired gases. This means, that with an open ventilation system the fraction of target gas supplied during the second ventilation stage can be controlled precisely. However, a disadvantage is the fact that the open ventilation system cannot be operated as cost-efficient as the closed ventilation system.

It should be noted, that in fact the steps comprising the second ventilation stage can be applied multiple times consecutively in order to make use of an overshoot. Usually, an overshoot within the second ventilation stage is not desired because the actual target gas concentration at the blood compartment might deviate too much from the desired target gas concentration which might put the patient's life at risk. However, the beginning of an overshoot might be induced during the second ventilation stage, whereas subsequently this overshoot is cushioned by counter-acting against the overshoot. Such a technique can be used to accelerate the change from the actual target gas concentration towards the desired gas concentration even further. From the field of control engineering this kind of overshoot technique is well-known, for example from the so-called PID-controller.

For some cases, the ventilation will finish after having reached the desired target gas concentration during the second ventilation stage or repetitions of the steps comprising the second ventilation stage which are applied one after the other. However, in most of the cases a third ventilation stage will be required in which a steady state of the desired target gas concentration is reached.

Therefore, according to a preferred aspect of the invention, the lung is ventilated in a third ventilation stage by setting a fraction of target gas, a fraction of re-breathed gas and a set of ventilation parameters, wherein the set of ventilation parameters yields a decreased ventilated lung volume compared to the second ventilation stage and wherein said setting results in the desired target gas concentration.

According to the explanations above, a closed ventilation system is again appropriate to be applied during the third ventilation stage.

In practice, the controlling means according to the invention for controlling the parameter varying means, the target gas varying means and the re-breathed gas varying means can comprise a switch for switching between a closed ventilation system (first ventilation stage) and an open ventilation system (second ventilation stage) and again a closed ventilation system (third ventilation stage).

According to another aspect of the invention the target gas is an anaesthetic agent. This means, that the invention applies to the field of anaesthesia where the concentration of the anaesthetic agent at the blood compartment has to be changed and where it is advantageous to reduce the time for performing such a change.

This means, that the invention can be applied both to a wash-in process of anaesthesia and to a wash-out process of anaesthesia. If the invention is applied to a wash-in process of anaesthesia the target gas supplied in the first ventilation stage is an anaesthetic agent corresponding to a state of shallow or no general anaesthesia and the target gas supplied in the second ventilation stage is an anaesthetic agent corresponding to a state of deeper general anaesthesia. On the other hand, if the invention is applied to a wash-out process of anaesthesia the target gas supplied in the first ventilation stage is an anaesthetic agent corresponding to a state of deeper general anaesthesia and the target gas supplied in the second ventilation stage is an anaesthetic agent corresponding to a state of shallow or no general anaesthesia.

The alveolar recruitment strategy is a well-tested method for temporarily increasing the ventilated lung volume. When applying the alveolar recruitment strategy the set of ventilation parameters during the second ventilation stage has to be adjusted accordingly. In general, the set of ventilation parameters of the first ventilation stage is based on a first peak inspiratory pressure and a first positive end-expiratory pressure. Furthermore, the set of ventilation parameters of the second ventilation stage is based on a time-varying second peak inspiratory pressure above the first peak inspiratory pressure and a time-varying second positive end-expiratory pressure above the first positive end-expiratory pressure. If a third ventilation stage as mentioned above is applied, the set of ventilation parameters of the third ventilation stage is based on a third peak inspiratory pressure, which is lower than the maximum of the time-varying second peak inspiratory pressure and a third positive end-expiratory pressure, which is lower or equal to the maximum of the time-varying second positive end-expiratory pressure.

With reference to FIG. 1, the set of ventilation parameters characterizing the first ventilation stage is applied in the beginning of the final recruitment maneuver (1 breath cycle comprising 3 breaths), the set of ventilation parameters characterizing the second ventilation stage is applied in the middle of the final recruitment maneuver (1 breath cycle comprising 10 breaths and 2 breath cycles in advance comprising each 3 breaths), and the set of ventilation parameters characterizing the third ventilation stage is applied in the end of the final recruitment maneuver (1 breath cycle comprising 3 breaths). It should be noted, that FIG. 1 and the corresponding description relate to one isolated example of an ARS only. Different ways of performing an ARS, in particular with respect to the number of breath cycles and breaths per cycle, can be employed within the method of the invention.

Another mode of ventilation for achieving an increased volume is a volume controlled ventilation. This mode has the advantage that the ventilated volume remains constant and that all changes of the lung status can be related to changes within the alveoli. In general, any possible mode of ventilation as well as any combination thereof can be applied according to the invention.

### BRIEF DESCRIPTION OF THE DRAWINGS

Other objects and features of the invention will become apparent by reference to the following specification, in which

FIG. 1 shows a sample plot of the airway pressures over time of a typical recruitment maneuver,

FIG. 2 shows a plot of the time constant (TAU) concept,

FIG. 3 shows a schematic representation of the underlying concept of the invention,

FIGS. 4 A,B,C show schematic representations of an anaesthesia system in a re-breathing and non-re-breathing mode according to the prior art,

FIG. 5 shows a plot of the expired anaesthetic fraction during start of anaesthesia,

FIG. 6 shows a plot of the expired anaesthetic fraction during end of anaesthesia,

FIG. 7 shows a plot of the gas kinetic during alveolar recruitment strategy (ARS),

FIG. 8 shows a schematic representation of the lung volumes for different gas volumes,

FIG. 9 shows a table of the combinations according to the invention between the set of ventilation parameters, the fraction of target gas and the fraction of re-breathed gas on the one hand and the different ventilation stages on the other hand,

FIG. 10 illustrates the operation of the alveolar recruitment strategy according to the prior art,

FIG. 11 illustrates the operation of the invention during a wash-in process, and

FIG. 12 illustrates the operation of the invention during a wash-out process.

### DETAILED DESCRIPTION OF THE INVENTION

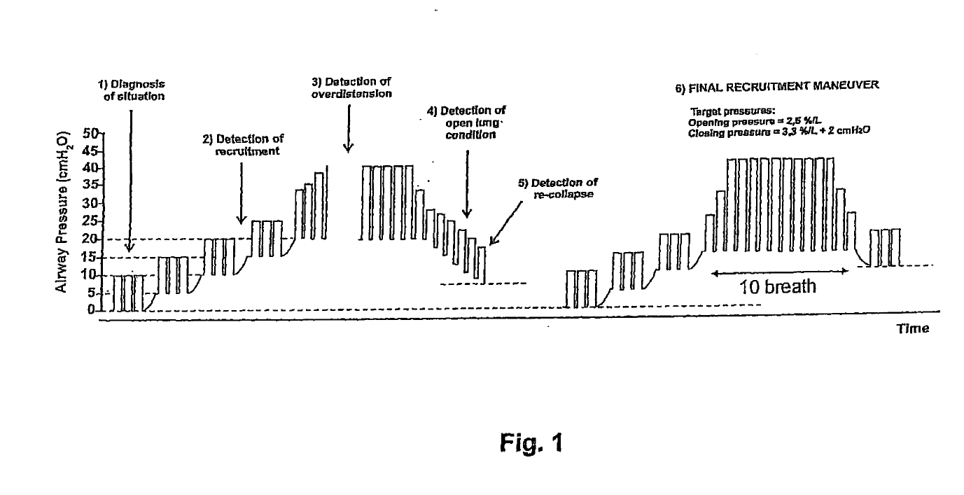

FIG. 1 has been explained in the introductory part.

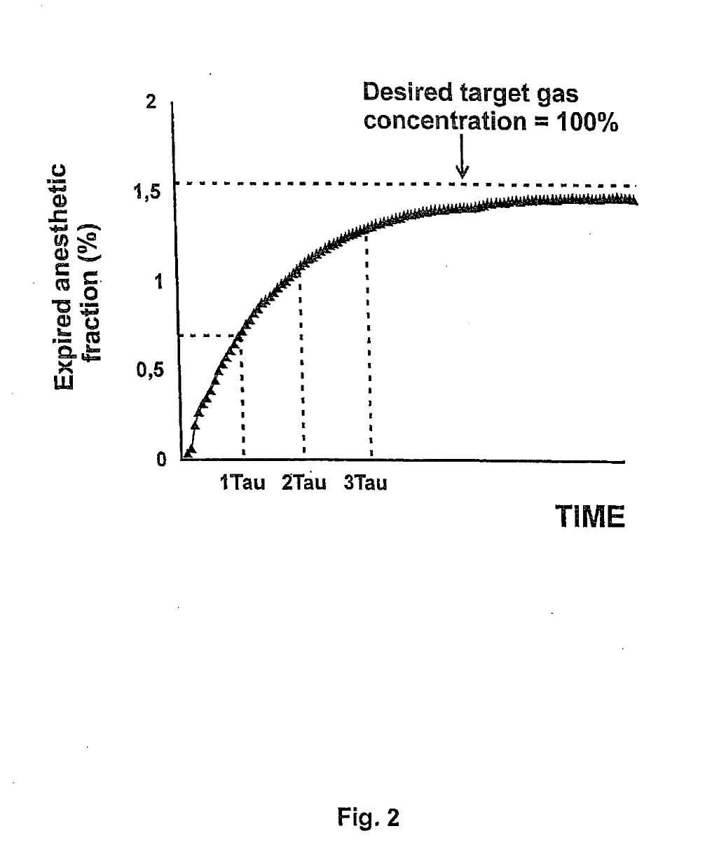

FIG. 2 shows a plot of the time constant (TAU) concept: The graphic shows the expired fraction of isofluorane being the target gas in this example against time using a semi-closed system. The first horizontal broken line indicates a concentration of 50% of the desired anaesthetic gas concentration. The corresponding first vertical broken line indicates the time required to reach this 50% concentration. This time period is called the time constant or TAU. After a time of 3×TAU more than 90% of the desired concentration is reached. The expired anaesthetic fraction represents the fraction of anaesthetic agent present in the gas being discarded from the patient. While this can be easily measured at the airway opening on-line and non-invasively, corresponding measurements of the target gas concentration of the blood compartment are considerably more difficult to perform. However, recordings of the expired anaesthetic fraction can be seen as a qualitative indication of the target gas concentration of the blood compartment, at least with respect to its variation.

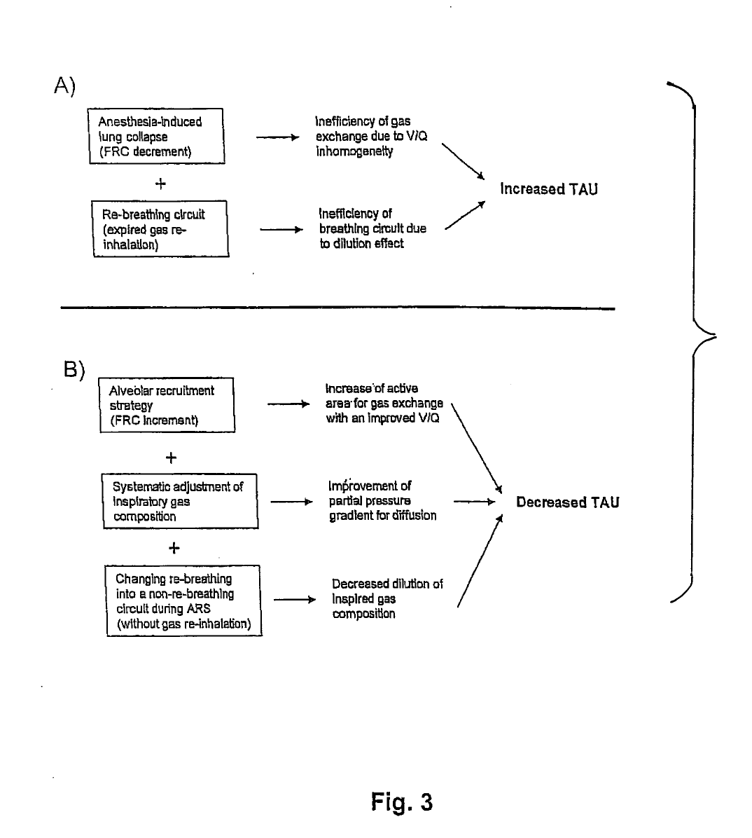

FIG. 3 shows a schematic representation of the underlying concept of the invention.

A) shows the concept of a standard anaesthesia machine. Both anaesthesia-induced lung collapse and re-breathing anaesthetic circuit increase TAU according to FIG. 2.

B) shows a new device according to the invention with a novel method and system to lower TAU. Due to the combination of alveolar recruitment strategy, a systematic adjustment of inspiratory gas composition and a changing of a closed ventilation system to an open ventilation system, i.e. changing from re-breathing to non-re-breathing during or before/after the alveolar recruitment strategy.

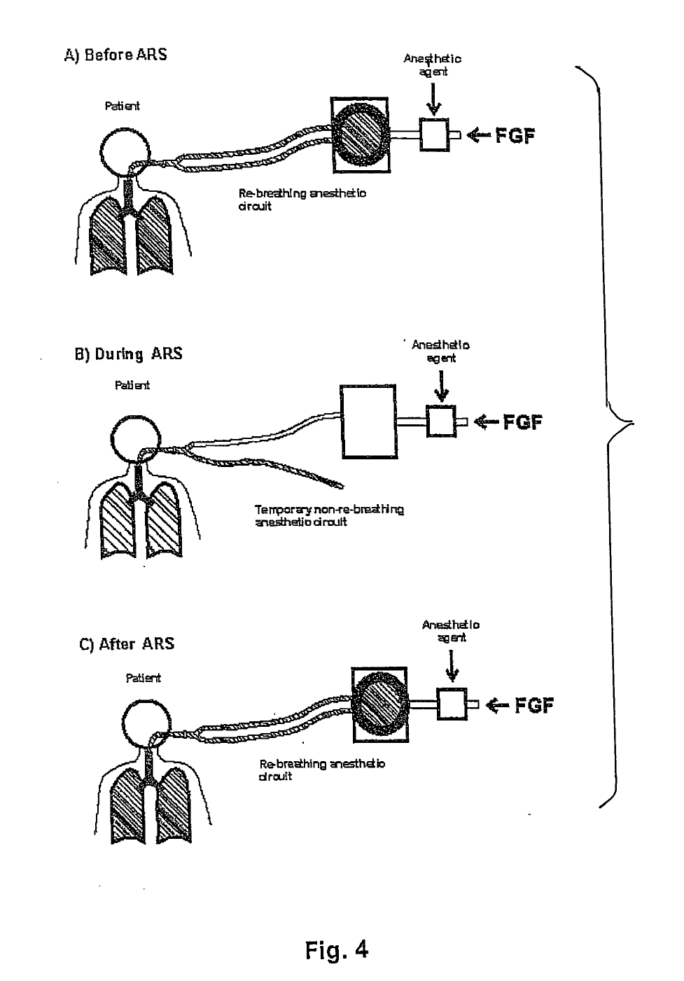

FIG. 4 shows a schematic representation of a typical anaesthesia system and its sequential modifications (A,B,C) according to the invention. While an alveolar recruitment maneuver is performed, the anaesthetic system is transformed from a re-breathing (A) into a non-re-breathing (so called “open”) system (B) where re-breathing of expired gas is eliminated. Afterwards, the anaesthetic circuit is transformed back into a re-breathing (so called “closed or semi-closed”) system (C).

A) shows a schematic representation of a re-breathing anaesthesia system. A fresh gas flow (FGF) is delivered into the patient through the inspired limb of the anaesthesia circuit. Expired gases return to the system through the expired limb of the anaesthesia circuit (striped area), diluting the fresh gas during the next inspiration (partially striped areas). This “dilution” effect increases the time constant (TAU) for any change in the concentration of the target gas within the inspired gas composition of anaesthetics.

B) shows a schematic representation of a non-re-breathing anaesthesia system. A fresh gas flow (FGF) is delivered into the patient through the inspired limb of the anaesthesia circuit while expired gases are discarded. During the next inspiration pure fresh gas is delivered to the patient. There is no “dilution” effect. Thus, the time constant (TAU) for any change in the concentration of the target gas within the inspired gas composition of anaesthetics is lower than in A.

C) shows the same schematic representation of a re-breathing anaesthesia system as under A. A fresh gas flow (FGF) is delivered into the patient through the inspired limb of the anaesthesia circuit. Expired gases return to the system through the expired limb of the anaesthesia circuit (striped area), diluting the fresh gas during the next inspiration (partially striped areas). This “dilution” effect increases the time constant (TAU) for any change in the concentration of the target gas within the inspired gas composition of anaesthetics.

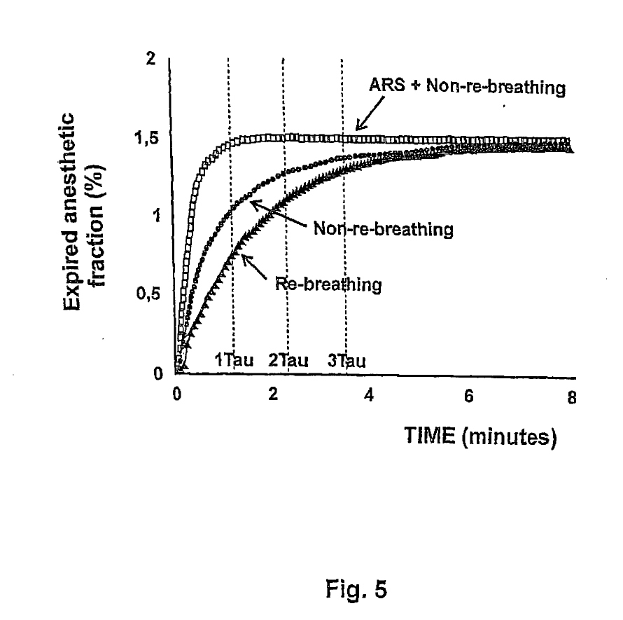

FIG. 5 shows a plot of the concentration of an anaesthetic agent in the expiratory gas composition, namely isofluorane which is the target gas in this example, during start of anaesthesia with wash-in of anaesthetic agent (desired concentration of the target gas in the expiratory gas composition=1.5%). The graphic shows the concentration of isofluorane in the expiratory gas composition against time using a common re-breathing “semi-closed” system (black triangles), an “open system” without re-breathing (black dots) and an alveolar recruitment maneuver (ARS) in conjunction with a non-re-breathing system (open squares). TAU is longer in the re-breathing circuit than in the two non-re-breathing systems. However, ARS in combination with a non-re-breathing decreases TAU even more, thus reaching the desired concentration of the target gas in the expiratory gas composition faster. Although, the concentration of the target gas in the expiratory gas composition was measured in the airway opening, a qualitatively similar result can be expected for the target gas concentration of the blood compartment.

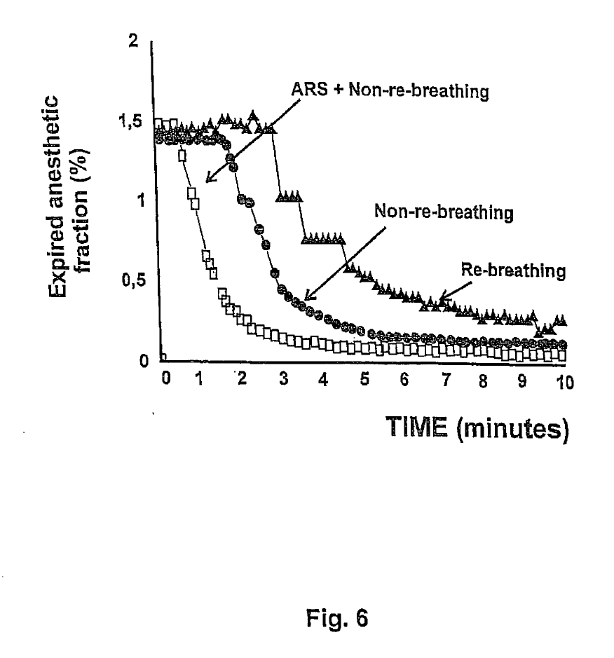

FIG. 6 shows a plot of the concentration of an anaesthetic agent in the expiratory gas composition, namely isofluorane which is the target gas in this example, during end of anaesthesia with a wash-out of anaesthetic agent (concentration of the target gas in the inspiratory gas composition =zero, desired concentration of the target gas in the expiratory gas composition=zero). The graphic shows expired isofluorane fraction against time using a common re-breathing “semi-closed” system (filled triangles), an “open” system without re-breathing (filled dots) and an alveolar recruitment maneuver (ARS) in conjunction with a non-re-breathing system (open squares). TAU is longer in the re-breathing circuit compared to the non-re-breathing systems. ARS applied in a non-re-breathing system decreases TAU even more, thus reaching the desired target gas concentration faster. Again, the concentration of the target gas in the expiratory gas composition gives a qualitative indication of the target gas concentration of the blood compartment, in particular, if an expired target gas fraction of 0% is present, the target gas concentration of the blood compartment is as well 0%.

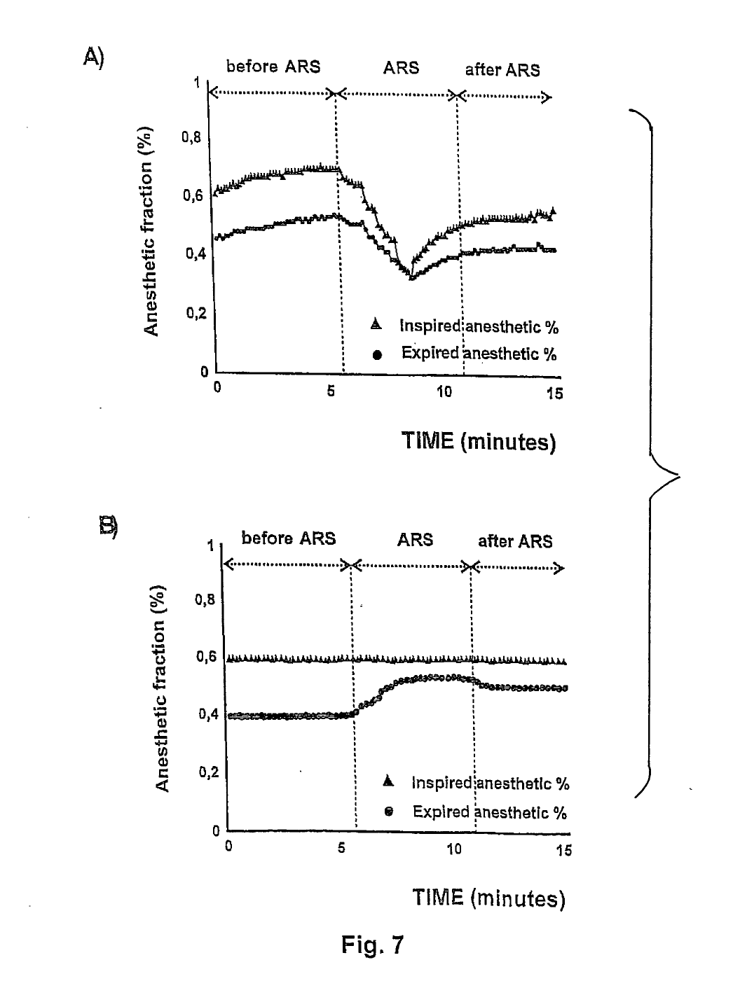

FIG. 7 shows a plot of the gas kinetic during alveolar recruitment strategy (ARS):

A) ARS performed in a semi-closed circuit, where re-breathing allows a dilution effect of anaesthetic gases (target gas). At the end, both inspiratory and expiratory gas compositions show target gas concentrations that reach a steady state at lower concentrations than before the ARS maneuver. Noticeably, the anaesthetic fraction of the inspired gas composition is reduced during the lung recruitment maneuver as a result of the dilution effect when increasing the lung volume. As a consequence, the anaesthetic fraction of the expired gas composition, and hence the actual target gas concentration of the blood compartment, is reduced as well. This effect is a problem within anaesthesia, e.g. inadvertent recovery or awareness of the patient, and it is the object of the invention to overcome this problem.

B) ARS without re-breathing, where a constant inspired gas composition is kept having a constant target gas concentration which corresponds to the desired target gas concentration of the blood compartment during and after the recruitment process. It can be noted that due to a better gas exchange obtained with a lung recruitment maneuver the difference between the target gas concentration in the inspiratory and in the expiratory gas composition is lower after ARS compared to the state before. This means that the invention makes anaesthesia more efficient.

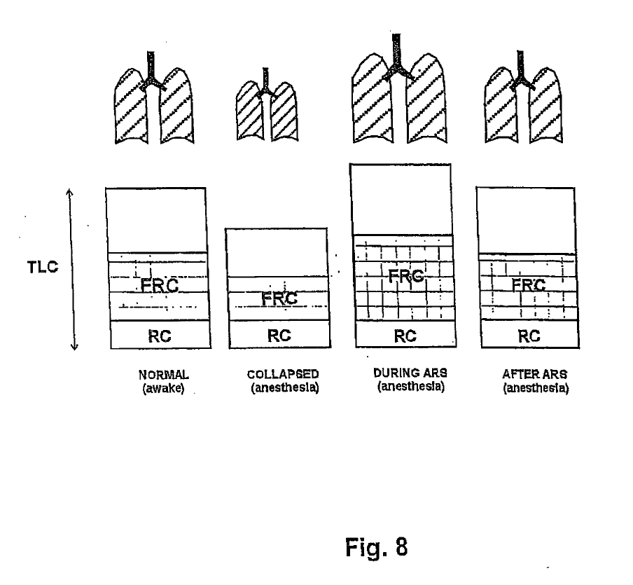

FIG. 8 shows a schematic representation of the lung volumes for different gas volumes in an awake patient, during anaesthesia as well as during and after the application of an alveolar recruitment strategy (ARS). Total lung capacity (TLC) is the volume of gas within lungs at end-inspiration. Functional residual capacity (FRC) is the volume of gas within lungs at end-expiration. It is reduced during anaesthesia due to lung collapse. The ARS restores normal lung volumes by recruiting previously collapsed lung units and is associated with normal gas exchange.

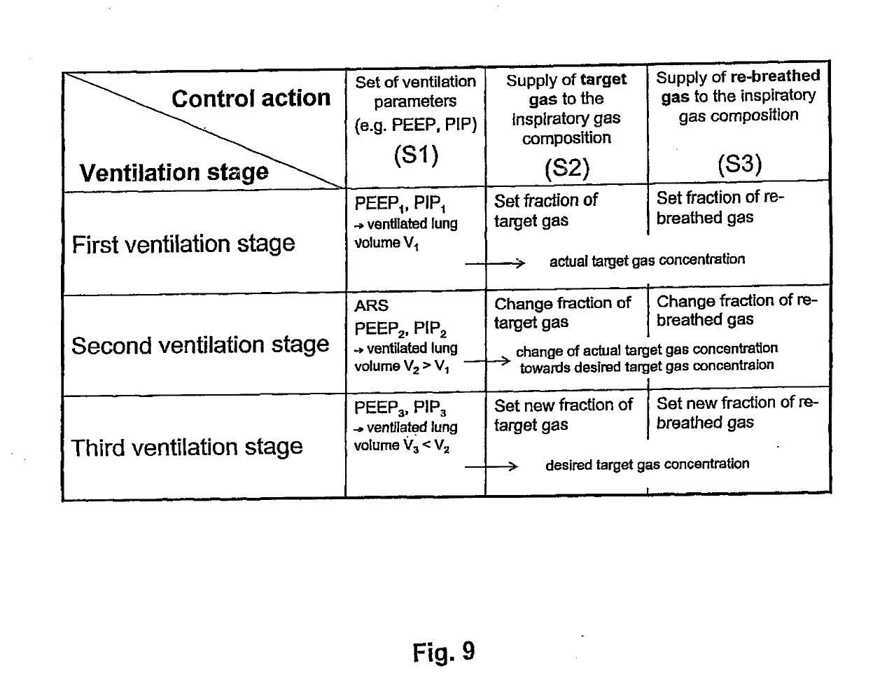

FIG. 9 shows a table of the combinations according to the invention between the set of ventilation parameters, the fraction of target gas and the fraction of re-breathed gas on the one hand and the different ventilation stages on the other hand. During the three ventilation stages the corresponding control actions or combinations thereof can be applied as already described above. The three control actions are based on the set of ventilation parameters (S1), the fraction of target gas supplied to the inspiratory gas composition (S2) and the fraction of re-breathed gas supplied to the inspiratory gas composition (S3). These three control actions can be used like control parameters known from the control theory to achieve the best performance of the change from the actual target gas concentration at the blood compartment to the desired target gas concentration at the blood compartment. This means that not necessarily all three actions have to be applied during one stage but that also only one or two control actions might be applied, where appropriate.

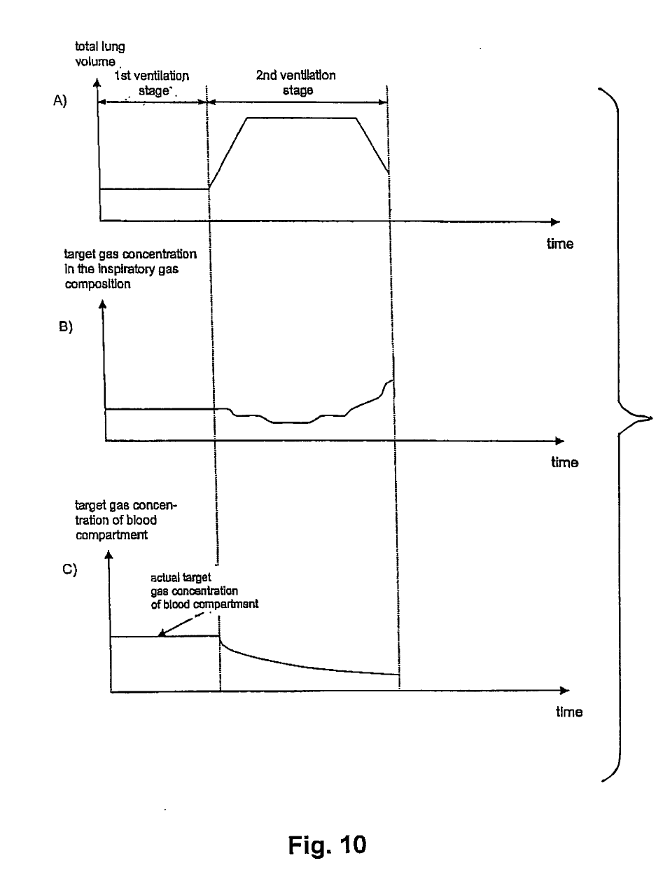

FIG. 10 illustrates the operation of the alveolar recruitment strategy according to the prior art. Shown are plots of the total lung volume, concentration of the target gas in the inspiratory gas composition and the target gas concentration of the blood compartment over the same time scale. In the first ventilation stage, before starting the lung recruitment maneuver, the total lung volume is small, while the target gas concentration of the inspiratory gas composition results in a certain target gas concentration of the blood compartment (steady state). Once the lung recruitment maneuver begins in the second ventilation stage, the lung volume increases. A conventional closed ventilation system is used so that a reduction of the target gas concentration of the blood compartment occurs during the second ventilation stage due to the dilution effect.

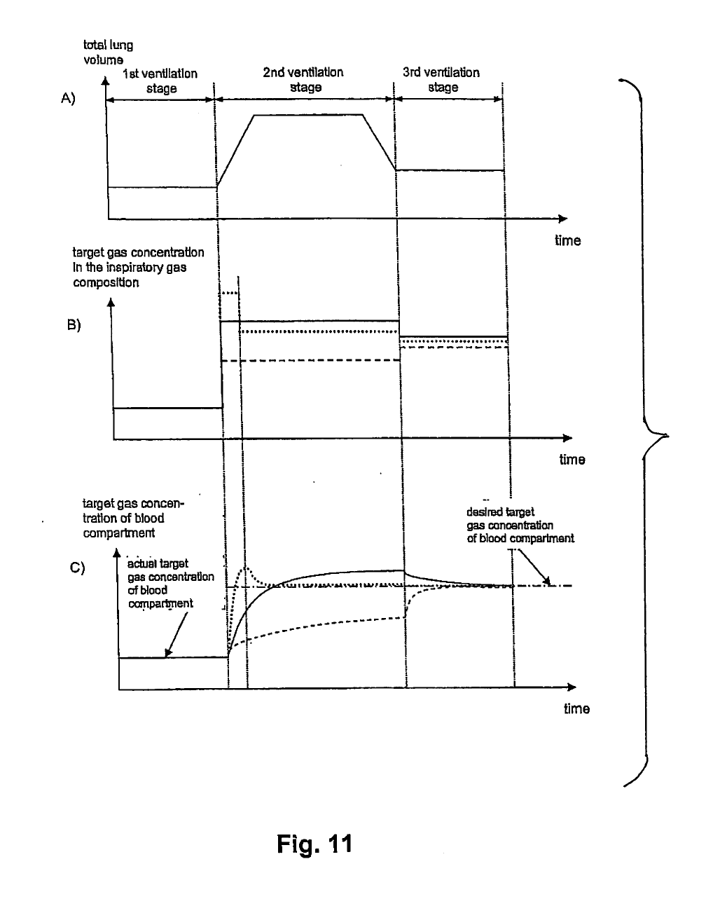

FIG. 11 illustrates the operation of the invention during a wash-in process. Shown are plots of the total lung volume, target gas concentration in the inspiratory gas composition and the target gas concentration of the blood compartment over the same time scale. In the first ventilation stage, before starting the lung recruitment maneuver, the total lung volume is small, while the target gas concentration of the inspiratory gas composition results in a certain target gas concentration of the blood compartment (steady state). Once the lung recruitment maneuver begins in the second ventilation stage, the total lung volume increases. According to the invention, the target gas concentration of the inspiratory gas composition within the second ventilation stage is modified by adjusting the fraction of target gas and the fraction of re-breathed gas supplied to the inspiratory gas composition in as such as to yield a change of the target gas concentration of the blood compartment towards the-desired target gas concentration. As depicted in FIG. 11c), this alteration of the target gas concentration of the blood compartment can be of various types, including an over-shoot. Similarly, the variation of the concentration of the target gas in the inspiratory gas composition can be of various types and can include multiple variations within the second ventilation stage. In the third ventilation stage the concentration of the target gas in the blood compartment reaches the desired target gas concentration of the blood compartment in a steady state.

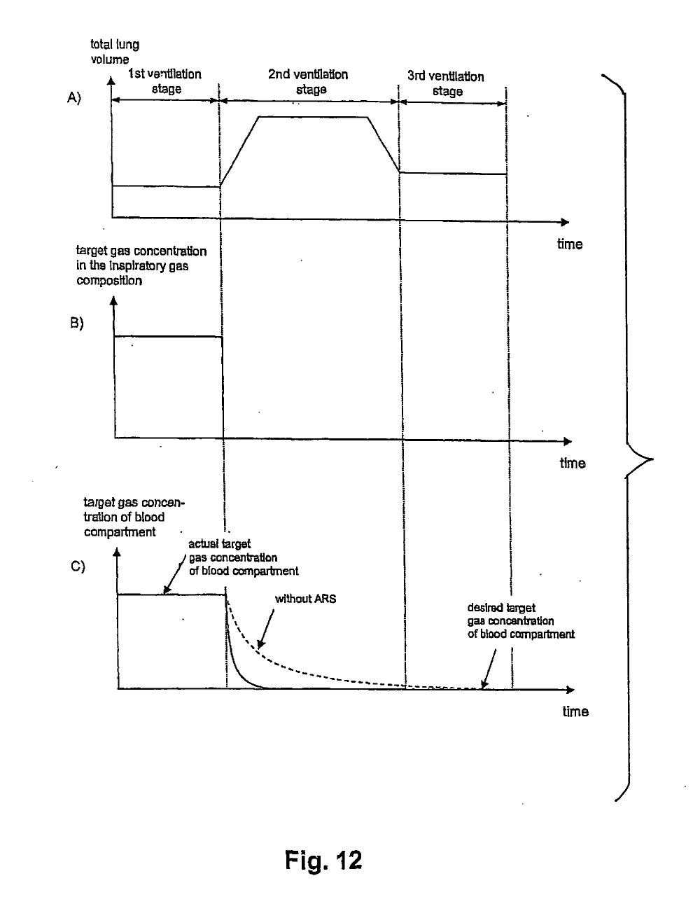

FIG. 12 illustrates the operation of the invention during a wash-out process. Shown are plots of the total lung volume, target gas concentration in the inspiratory gas composition and the target gas concentration of the blood compartment over the same time scale. The target gas shall be removed completely from the blood compartment. In the first ventilation stage, before starting the lung recruitment maneuver, the total lung volume is small, while the target gas concentration of the inspiratory gas composition results in a certain target gas concentration of the blood compartment (steady state). Once the lung recruitment maneuver begins in the second ventilation stage, the lung volume increases. According to the invention, the concentration of the target gas within the second ventilation stage is modified by adjusting the fraction of target gas and the fraction of re-breathed gas supplied to the inspiratory gas composition in as such as to yield a decrease of the target gas concentration of the blood compartment. Preferably, in a wash-out process the concentration of the target gas within the inspiratory gas composition is 0%. A wash-out process of the target gas without ARS would result in a slower withdrawal of the target gas from the blood compartment.

We claim:

1. Method for changing the concentration of a target gas at the blood compartment of a patient's lung from an actual target gas concentration to a desired target gas concentration during artificial ventilation with an inspiratory gas composition by a respirator being controlled via a set of ventilation parameters, by varying
the fraction of the target gas supplied to the inspiratory gas composition, and/or
the fraction of the re-breathed gas supplied to the inspiratory gas composition, and/or
the set of ventilation parameters being responsible for the ventilated lung volume,
wherein
a) the lung is ventilated in a first ventilation stage by setting a fraction of target gas, a fraction of re-breathed gas and a set of ventilation parameters, wherein said setting results in the actual target gas concentration, and
b) the lung is ventilated in a second ventilation stage in which at least once
the set of ventilation parameters is varied for yielding an increased ventilated lung volume compared to the first ventilation stage and
on the basis of the increased ventilated lung volume the fraction of target gas and/or the fraction of re-breathed gas is varied such that the target gas concentration is changed towards the desired target gas concentration.

2. Method according to claim 1, wherein the lung is ventilated in a third ventilation stage by setting a fraction of target gas, a fraction of re-breathed gas and a set of ventilation parameters, wherein the set of ventilation parameters yields a decreased ventilated lung volume compared to the second ventilation stage and wherein said setting results in the desired target gas concentration.

3. Method according to claim 1, wherein the target gas is an anaesthetic agent.

4. Method according to claim 3, wherein during a wash-in process of anaesthesia the target gas supplied in the first ventilation stage is an anaesthetic agent corresponding to a state of shallow or no general anaesthesia and the target gas supplied in the second ventilation stage is an anaesthetic agent corresponding to a state of deeper general anaesthesia.

5. Method according to claim 3, wherein during a wash-out process of anaesthesia the target gas supplied in the first ventilation stage is an anaesthetic agent corresponding to a state of deeper general anaesthesia and the target gas supplied in the second ventilation stage is an anaesthetic agent corresponding to a state of shallow or no general anaesthesia.

6. Method according to claim 1, wherein the set of ventilation parameters of the first ventilation stage is based on a first peak inspiratory pressure and a first positive end-expiratory pressure.

7. Method according to claim 1, wherein the set of ventilation parameters of the second ventilation stage is based on a time-varying second peak inspiratory pressure above the first peak inspiratory pressure and a time-varying second positive end-expiratory pressure above the first positive end-expiratory pressure.

8. Method according to claim 2, wherein the set of ventilation parameters of the third ventilation stage is based on a third peak inspiratory pressure, which is lower than the maximum of the time-varying second peak inspiratory pressure and a third positive end-expiratory pressure, which is lower or equal to the maximum of the time-varying second positive end-expiratory pressure.

9. Apparatus for changing the concentration of a target gas at the blood compartment of a patient's lung from an actual target gas concentration to a desired target gas concentration during artificial ventilation with an inspiratory gas composition by a respirator being controlled via a set of ventilation parameters, comprising
target gas varying means for varying the fraction of the target gas supplied to the inspiratory gas composition,
re-breathed gas varying means for varying the fraction of the re-breathed gas supplied to the inspiratory gas composition,
parameter varying means for varying the ventilation parameters being responsible for the ventilated lung volume, and
controlling means for controlling the target gas varying means, the re-breathed gas varying means and the parameter varying means such that
a) the lung is ventilated in a first ventilation stage by setting a fraction of target gas, a fraction of re-breathed gas and a set of ventilation parameters, wherein said setting results in the actual target gas concentration, and
b) the lung is ventilated in a second ventilation stage in which at least once
the set of ventilation parameters is varied for yielding an increased ventilated lung volume compared to the first ventilation stage and
on the basis of the increased ventilated lung volume the fraction of target gas and/or the fraction of re-breathed gas is varied such that the target gas concentration is changed towards the desired target gas concentration.

10. Apparatus according to claim 9, wherein the lung is ventilated in a third ventilation stage by setting a fraction of target gas, a fraction of re-breathed gas and a set of ventilation parameters, wherein the set of ventilation parameters yields a decreased ventilated lung volume compared to the second ventilation stage and wherein said setting results in the desired target gas concentration.

11. Apparatus according to claim 9, wherein the target gas is an anaesthetic agent.

12. Apparatus according to claim 11, wherein during a wash-in process of anaesthesia the target gas supplied in the first ventilation stage is an anaesthetic agent corresponding to a state of shallow or no general anaesthesia and the target gas supplied in the second ventilation stage is an anaesthetic agent corresponding to a state of deeper general anaesthesia.

13. Apparatus according to claim 11, wherein during a wash-out process of anaesthesia the target gas supplied in the first ventilation stage is an anaesthetic agent corresponding to a state of deeper general anaesthesia and the target gas supplied in the second ventilation stage is an anaesthetic agent corresponding to a state of shallow or no general anaesthesia.

14. Apparatus according to claim 9, wherein the set of ventilation parameters of the first ventilation stage is based on a first peak inspiratory pressure and a first positive end-expiratory pressure.

15. Apparatus according to claim 9, wherein the set of ventilation parameters of the second ventilation stage is based on a time-varying second peak inspiratory pressure above the first peak inspiratory pressure and a time-varying second positive end-expiratory pressure above the first positive end-expiratory pressure.

16. Apparatus according to claim 10, wherein the set of ventilation parameters of the third ventilation stage is based on a third peak inspiratory pressure, which is lower than the maximum of the time-varying second peak inspiratory pressure and a third positive end-expiratory pressure, which is lower or equal to the maximum of the time-varying second positive end-expiratory pressure.
